(19) United States
(12) Patent Application Publication
(10) Pub. No.: US 2017/0069086 A1
(43) Pub. Date: Mar. 9, 2017

(54) METHOD AND DEVICE FOR CONSTRUCTING BRAIN TEMPLATES

(71) Applicant: Xuanwu Hospital Capital Medical University, Beijing (CN)

(72) Inventors: Kuncheng LI, Beijing (CN); Peipeng LIANG, Beijing (CN); Defeng WANG, Hong Kong (CN); Lin SHI, Hong Kong (CN)

(73) Assignee: Xuanwu Hospital Capital Medical University, Beijing (CN)

(21) Appl. No.: 15/256,807

(22) Filed: Sep. 6, 2016

(30) Foreign Application Priority Data
Sep. 9, 2015 (CN) 201510570129.0

(51) Int. Cl. G06T 7/00; A61B 5/055; G01R 33/58; A61B 5/00

(52) U.S. Cl. CPC G06T 7/0014; A61B 5/0042; A61B 5/055; G06T 7/0036; G01R 33/58; A61B 2576/026; G06T 2207/30016; G06T 2207/10088; G06T 2207/20076; G06T 2207/20036

(57) ABSTRACT

Embodiments of the present disclosure provide a method for constructing brain templates. First, thousands of brain MRI images are collected. Then, the collected brain MRI images are preprocessed and normalized to construct brain templates. Further, the constructed brain templates include brain templates corresponding to different age and gender groups and brain tissue probability maps corresponding to different age and gender groups. Therefore, embodiments of the present disclosure can construct brain templates by using the large sample size of brain MRI data, and support clinical applications and brain researches.

### CROSS REFERENCE TO RELATED APPLICATIONS

The present disclosure relates to medical image processing techniques, and This application claims the benefit of priority from Chinese Patent Application No. 201510570129.0, entitled “Method and Device for Constructing Brain Templates” and filed on Sep. 9, 2015, the entire content of which is hereby incorporated by reference.

### TECHNICAL FIELD

The present disclosure relates to medical image processing techniques, and more particularly, to a method and device for constructing brain templates.

### BACKGROUND

Despite the rapid progress of brain and cognition researches, less is known about the origin of consciousness, thought, as well as the material basis of intelligence and creativity. In particular, with the growing of neurological and psychiatric diseases which severely affects human healthy, it is of urgent to deeply understand the structure of the brain, e.g., what is the structure of the healthy brain, what is the developmental and degenerative law of the brain structure, etc. As the important infrastructure of brain science, construction of brain templates is one of the core issues to deal with the abovementioned problems.

### SUMMARY OF THE DISCLOSURE

In view of this, embodiments of the present disclosure provide a method and device for constructing brain templates, which can construct brain templates based on large data samples.

A method for constructing brain templates includes:

collecting brain magnetic resonance imaging (MRI) images; and

preprocessing and normalizing the brain MRI images to construct brain templates.

The brain templates include brain templates corresponding to different age ranges and genders (male/female) and brain tissue probability maps corresponding to the different age ranges and genders.

The number of the brain MRI images is larger than one thousand. The age of subjects is ranged from 20 to 75 years old, and the gender of the subjects comprises male and female.

The method for preprocessing and normalizing the brain MRI images includes:

correcting the bias field of the brain MRI images and adjusting brain orientation;

partitioning the brain MRI images into different age and gender groups;

performing spatial normalization and strength distribution normalization for the grouped brain MRI images;

denoising the brain MRI images after the strength distribution normalization processing to construct the brain templates.

The spatial normalization processing adopts a histogram registration method.

The histogram registration method comprises a masked, differential homeomorphism transformation based, nonlinear registration.

Brain templates are constructed by applying a weighted average template algorithm based on kernel regression to the brain MRI images of different age groups.

Brain templates corresponding to the different age and gender groups comprise 24 brain templates, which are constructed for different age groups ranging from 20 to age 75 years old at a 5 years interval and are separately constructed for male and female in each age group.

A device for constructing brain templates includes: a storage and a processor;

wherein the processor is to execute machine readable instructions in the storage to:

collect brain magnetic resonance imaging MRI images; and

preprocess and normalize the brain MRI images to construct brain templates.

It can be seen from the above scheme that, with the method for constructing brain templates provided by embodiments of the present disclosure, thousands of brain MRI images are firstly collected. Then, the collected brain MRI images are preprocessed and normalized to construct brain templates. The constructed brain templates include brain templates corresponding to different age and gender groups and brain tissue probability maps corresponding to the different age and gender groups. Therefore, embodiments of the present disclosure offer a new method for constructing the brain templates using the large data samples, and support clinical applications and brain researches.

### BRIEF DESCRIPTION OF THE DRAWINGS

FIG. 1 is a flow chart illustrating the method for constructing brain templates in accordance with various embodiments of the present disclosure;

FIG. 2 is a diagram illustrating twelve brain templates provided by various embodiments of the present disclosure;

FIG. 3 is a diagram illustrating brain tissue probability maps in the brain templates in accordance with various embodiments of the present disclosure;

FIG. 4 is a flow chart illustrating the method for constructing brain templates in accordance with various embodiments of the present disclosure;

FIG. 5 is a diagram illustrating the collected original brain MRI images in accordance with various embodiments of the present disclosure;

FIG. 6 is a diagram illustrating the comparison between the constructed brain templates and existing brain templates in accordance with various embodiments of the present disclosure; and

FIG. 7 is a diagram illustrating structure of a device for constructing brain templates in accordance with various embodiments of the present disclosure.

### DETAILED DESCRIPTION OF THE EMBODIMENTS

To make the objective, technical solution as well as the merits of the present disclosure more clearly, the present disclosure is described in details with reference to the accompanying figures.

There have been some studies about the brain template, all of which construct brain templates by collecting and processing brain MRI images. However, these brain templates are constructed by using a postmortem brain, or multiple scans of an individual brain, or a limited number of samples of brain MRI images. When applying these brain templates to subjects with different ages, genders and races, some potential bias may occur, which has been one of the main problems restricting the brain structure and function related studies. Particularly, there are no Chinese brain templates so far. Therefore, in the clinical applications concerning Chinese population, only Caucasian brain templates are available to determine whether the brain shown in the collected brain image is healthy or not. Additionally, in the brain imaging studies using Chinese subjects, the Chinese brain MRI images have to be registered to the Caucasian brain templates to report the brain activation. Due to the significant brain morphological difference between east Asian and Caucasian, this may lead to inaccurate diagnosis and bias, even error in functional and structural localization. Therefore, it is urgent to construct the brain template based on large scale and multi-center MRI data samples to provide objective and accurate brain morphological characteristics of different ages and genders and then support the validation and comparison of brain developmental and aging theories.

It can be seen from above that the amount of the samples used to construct the existing brain templates is relatively small and thus the representativeness is weak. In particular, there may be errors during the brain functional and structural localization as well as disease diagnosis concerning Chinese population based on the existing brain templates. Therefore, with the proposed method provided in embodiments of the present disclosure, thousands of brain MRI images are collected, and then, the collected brain MRI images are preprocessed and normalized to construct the brain template.

Further, the constructed brain templates include brain templates corresponding to different ages and genders as well as brain tissue probability maps corresponding to the different ages and genders.

In embodiments of the present disclosure, brain templates are constructed based on thousands of brain MRI images, i.e., a large scale and multi-center data samples. Thus, the constructed brain templates may accurately reflect the brain morphological characteristics of different ages and genders, and may overcome the inadequacy of the existing brain templates.

In embodiments of the present disclosure, the constructed brain templates correspond to different age and gender groups. Furthermore, the brain template of any age and gender can be customized. That is, the brain templates can be customized directly according to patients' or subjects' age and gender in the clinical applications or brain researches.

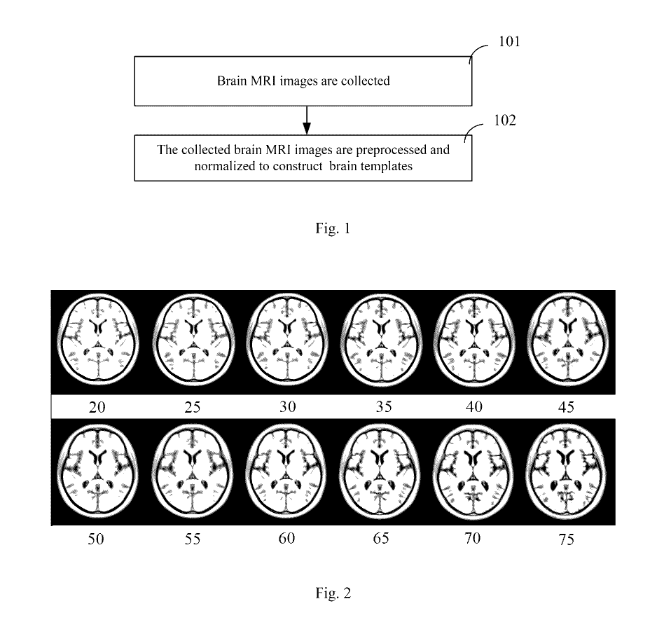

FIG. 1 is a flow chart illustrating the method for constructing brain templates in accordance with various embodiments of the present disclosure. This method includes the following blocks.

At block 101, brain MRI images are collected.

In embodiments of the present disclosure, the brain MRI images are collected from a large scale of samples including thousands of subjects. And the number of the brain MRI images may be more than one thousand, such as three thousands. Further, the brain MRI images may be brain MRI images of human beings, or animals. If the brain MRI images are from human beings, these brain MRI images may be the brain MRI images of any races, such as Asians. For example, the age of subjects may range from 20 to 75 years old, and the gender is male or female.

At block 102, the collected brain MRI images are preprocessed and normalized to construct brain templates.

The steps for the preprocessing and normalization of brain MRI images are as follows.

First, the bias field correction and the adjustment of brain orientations are performed. The collected brain MRI images are set in the center of the coordinate space and the brain orientation is set to be positive.

In this process, the bias field correction is executed by using Gaussian filtering and B-spline interpolation.

Second, the collected brain MRI images are partitioned into different age and gender groups. Then, spatial normalization and strength distribution normalization are executed in sequence for the grouped brain MRI images.

In this process, spatial normalization is performed based on the existing basal brain templates, such as the MNI152 brain templates. Strength distribution normalization is also performed based on the existing basal brain templates, such as the Colin27 brain templates.

Third, after the strength distribution normalization processing, the brain MRI images are denoised to construct brain templates.

In this process, the brain MRI images are denoised by using the spatial Gaussian filtering.

Spatial normalization is performed based on image registration. The accuracy of image registration may to a big extent affect the clarity and accuracy of the constructed brain templates. Linear registration and nonlinear registration of low degree-of-freedom cannot be used as they cannot accurately estimate the difference between different individuals. Therefore, embodiments of the present disclosure adopt a high degree-of-freedom and high-precision, differential homeomorphism transformation based nonlinear registration method. Particularly, in order to reduce the impact of the background noises and improve convergence speed, embodiments of the present disclosure design a masked, differential homeomorphism transformation based, nonlinear registration method. That is, brain tissue areas are extracted from the reference image space using a brain extraction algorithm and a brain mask is then made based on these brain tissue areas. When an individual MRI image is registered to a reference brain MRI image, only the voxels within the mask are calculated, which may reduce the possibility of slow convergence speed and local minimization due to the impact of the background noises in the registration process. The whole spatial normalization processing adopts the histogram registration method.

According to the above method, the steps for building brain templates are as follows.

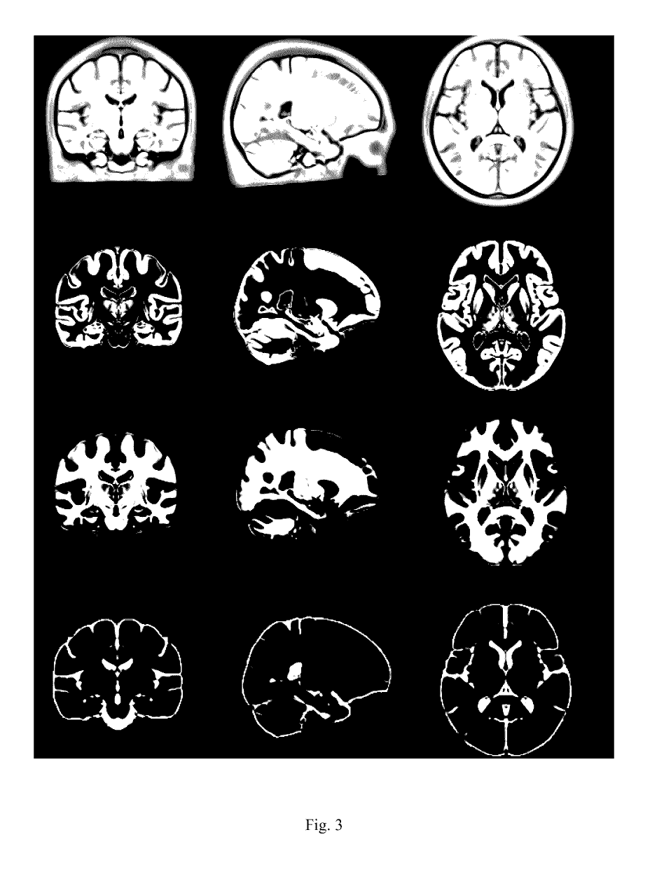

According to the age of subjects, the brain templates can be constructed for different age groups ranging from 20 to 75 years old at a 5 years interval and the brain templates can be separately constructed for male and female in each age group. FIG. 2 is a diagram illustrating the 12 brain templates provided by various embodiments of the present disclosure. The corresponding brain space is constructed using a group image registration method. In order to make the constructed brain templates reflect the characteristics of a specific age group and vary smoothly in time and space, a kernel regression based, weighted average template algorithm is adopted to construct the brain templates for a specific age group. Each brain template corresponding to a specific age group may to some extent accurately reflect the characteristics of the age. The weight of the individual brain in the construction of the brain templates is calculated based on the individual age. The individual, age of which is close to the age of the brain templates, is allocated with a larger weight. In this way, the bias caused by non-uniform distribution of age can be reduced.

In embodiments of the present disclosure, the constructed brain templates may also include corresponding brain tissue probability maps. As shown in FIG. 3, the first line shows whole brain images, the second line shows grey matter probability map, the third line shows white matter probability map and the fourth line shows Cerebrospinal Fluid (CSF) probability map.

Embodiments of the present disclosure can be further described in a specific example.

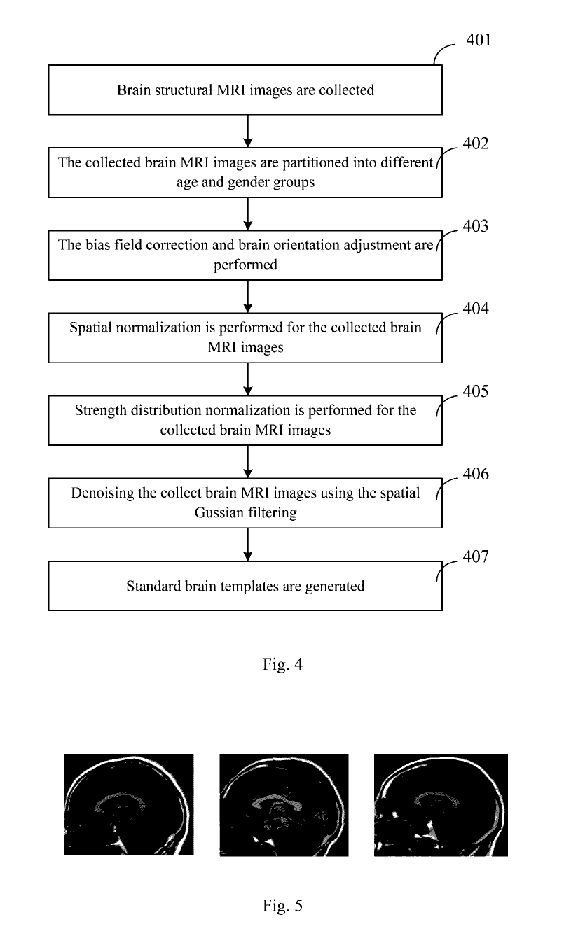

FIG. 4 is a flow chart illustrating the method for constructing brain templates in accordance with various embodiments of the present disclosure. The following blocks are included.

At block 401, MRI data collection is performed.

At this block, as shown in FIG. 5, original brain structural MRI images are collected.

At block 402, the brain MRI images collected are partitioned into different age and gender groups.

At block 403, the bias field correction and brain orientation adjustment are performed.

At block 404, the spatial normalization is performed for the brain MRI images.

At this block, the spatial normalization processing may adopt a masked, differential homeomorphism transformation based, nonlinear registration method.

At block 405, strength distribution normalization is performed for the brain MRI images.

At block 406, the brain MRI images are denoised using the spatial Gaussian filtering.

At block 407, brain templates are constructed.

Brain templates, as shown in FIGS. 2 and 3, are constructed by using a kernel regression based, weighted average template algorithm.

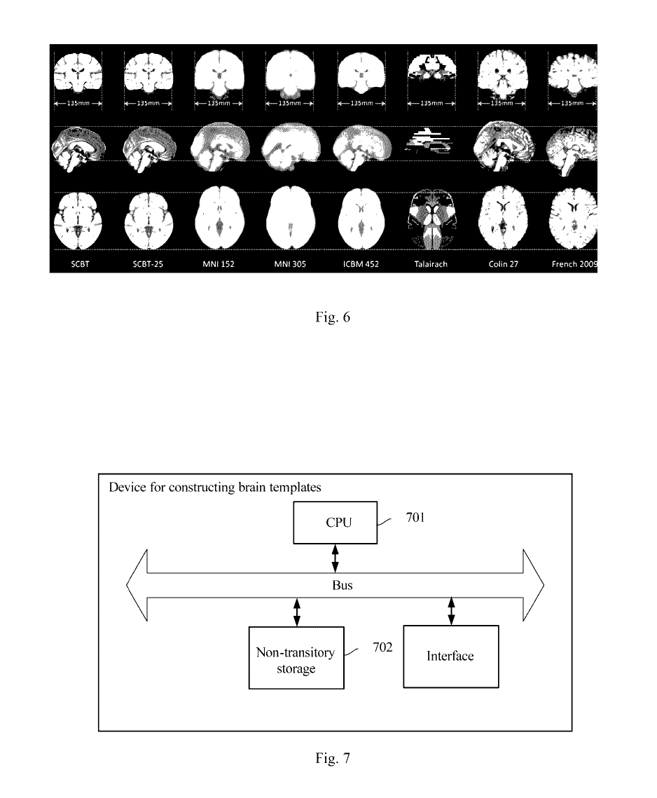

FIG. 6 is a diagram illustrating the comparison between the constructed brain templates and existing brain templates in accordance with various embodiments of the present disclosure. The last six columns denote the structure of existing different brain templates. The first and second columns show the structure of the constructed brain templates using the method provided in embodiments of the present disclosure. The first line of FIG. 6 shows brain templates in the coronal view, the second line shows brain templates in the sagittal view, and the third line shows brain templates in the axial view.

FIG. 7 is a schematic diagram illustrating structure of a device for constructing brain templates in accordance with various examples of the present disclosure.

The device may include: a CPU 701 and a non-transitory storage 702.

The non-transitory storage 702 may be configured to store machine readable instructions.

The CPU 701 may communicate with the non-transitory storage 702 to read and execute the machine readable instructions on the non-transitory storage 702, to

collect brain magnetic resonance imaging MRI images; and

preprocess and normalize the brain MRI images to construct brain templates.

The CPU 701 is further to execute the machine readable instructions in the non-transitory storage 702 to

correct the bias field of the brain MRI images and adjust brain orientation;

partition the brain MRI images into different age and gender groups;

perform spatial normalization and strength distribution normalization for the brain MRI images grouped; and

denoise the brain MRI images after the strength distribution normalization processing to construct the brain templates.

What has been described and illustrated herein are examples of the disclosure along with some variations. The terms, descriptions and figures used herein are set forth by way of illustration only and are not meant as limitations. Many variations are possible within the scope of the disclosure, which is intended to be defined by the following claims—and their equivalents—in which all terms are meant in their broadest reasonable sense unless otherwise indicated.

What is claimed is:

1. A method for constructing brain templates, comprising:
collecting brain magnetic resonance imaging MRI images; and
preprocessing and normalizing the brain MRI images to construct brain templates.

2. The method according to claim 1, wherein the brain templates comprise: brain templates corresponding to different age and gender groups.

3. The method according to claim 2, wherein the brain templates further comprises: brain tissue probability maps corresponding to different age and gender groups.

4. The method according to claim 1, wherein the number of brain MRI images is larger than one thousand.

5. The method according to claim 1, wherein the age of subjects of the brain MRI images are ranged from 20 to 75 years old.

6. The method according to claim 1, wherein the gender of subjects of the brain MRI images comprise male and female.

7. The method according to claim 1, wherein the preprocessing and normalizing the brain MRI images comprises:
correcting the bias field of the brain MRI images and adjusting brain orientation;
partitioning the brain MRI images into different age and gender groups;
performing spatial normalization and strength distribution normalization for the brain MRI images grouped; and
denoising the brain MRI images after the strength distribution normalization processing to construct the brain templates.

8. The method according to claim 7, wherein the spatial normalization adopts a histogram registration method.

9. The method according to claim 8, wherein the histogram registration method is a masked, differential homeomorphism transformation based, nonlinear registration method.

10. The method according to claim 8, wherein brain templates are constructed by using a kernel regression based, weighted average template algorithm for different age and gender groups.

11. The method according to claim 2, wherein the brain templates corresponding to the different age and gender groups comprise 24 brain templates, which are constructed for different age groups ranging from 20 to 75 years old at a 5 years interval and are constructed separately for male and female in each age group.

12. A device for constructing brain templates, comprising: a storage and a processor, wherein the processor is to execute machine readable instructions in the storage to:
collect brain magnetic resonance imaging MRI images; and
preprocess and normalize the brain MRI images to construct brain templates.

13. The device according to claim 12, wherein the brain templates comprise: brain templates corresponding to different age and gender groups.

14. The device according to claim 13, wherein the brain templates further comprises: brain tissue probability maps corresponding to different age and gender groups.

15. The device according to claim 12, wherein the number of brain MRI images is larger than one thousand.

16. The device according to claim 12, wherein the gender of subjects of the brain MRI images comprise male and female.

17. The device according to claim 12, wherein the processor is further to execute the machine readable instructions in the storage to:
correct the bias field of the brain MRI images and adjust brain orientation;
partition the brain MRI images into different age and gender groups;
perform spatial normalization and strength distribution normalization for the brain MRI images grouped; and
denoise the brain MRI images after the strength distribution normalization processing to construct the brain templates.

18. The device according to claim 17, wherein the spatial normalization adopts a histogram registration method.

19. The device according to claim 18, wherein the histogram registration method is a masked, differential homeomorphism transformation based, nonlinear registration method.

20. The device according to claim 18, wherein brain templates are constructed by using a kernel regression based, weighted average template algorithm for different age and gender groups.
